(19) United States
(12) Patent Application Publication
(10) Pub. No.: US 2012/0058977 A1
(43) Pub. Date: Mar. 8, 2012

(54) PHARMACEUTICAL COMPOSITIONS AND METHODS FOR TREATING TUBERCULOSIS

(75) Inventors: Robert Van Der Geize, Veendam (NL); Lubbert Dijkhuizen, Zuidlaren (NL); Martin Ostendorf, Oss (NL); Peter Van Der Meijden, Oss (NL)

(73) Assignee: Rijksuniversiteit Groningen, Groningen (NL)

(21) Appl. No.: 13/202,765

(22) PCT Filed: Feb. 23, 2010
(86) PCT No.: PCT/EP2010/052299

§ 371 Date: Nov. 9, 2011

(30) Foreign Application Priority Data
Feb. 23, 2009 (EP) 091534669

(51) Int. Cl. A61K 31/59; A61K 31/122; A61P 31/06; A61K 31/045; C07C 401/00; A61P 31/04; C07C 49/443; C07C 35/32

(52) U.S. Cl. 514/167; 568/374; 514/691; 568/819; 514/729; 552/653

(57) ABSTRACT

A pharmaceutical composition for the treatment of a disease caused by a bacterium that belongs to the group of nocardioform actinomycetes, said composition comprising an effective amount of a compound selected from compound I, (+)-compound II, (−)-compound II, compound III, or mixtures thereof.

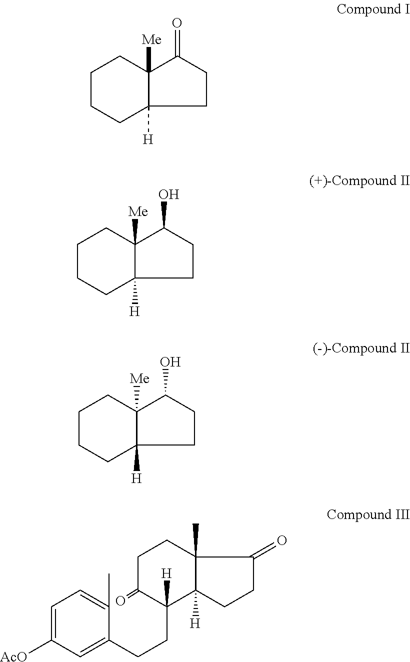

### FIELD OF INVENTION

The present invention pertains to a pharmaceutical composition for use in the treatment of a disease caused by a bacterium that belongs to the group of nocardioform actinomycetes. Furthermore, the invention also pertains to a method for treating a subject suffering from a disease caused by a bacterium that belongs to the group of nocardioform actinomycetes. Finally, the invention provides a new compound.

### BACKGROUND OF THE INVENTION

Within the class Actinobacteria there is an order of bacteria called Actinomycetales, commonly referred to as actinomycetes. Bacteria that belong to this order are filamentous gram positive bacteria (several species however have complex cell wall structures which makes classic Gram staining less- or even unsuitable, for example as is the case with many species that belong to the Actinomycetales family Mycobacteriaceae) with a high G+C content. They are best known as soil dwelling organisms, although various strains inhabit plants and animals, including humans. They produce resistant spores which are often attached to aerial mycelium or hyphae. Actinomycetes play an important role in the decomposition of organic material. Several species are used in industry and pharma-research because of their typical properties.

Most actinomycetes are non-pathogenic for animals, including humans. However, within the many suborders of the actinomycetes (i.a. Streptosporangineae, Micrococcineae, Streptomycineae and Frankineae) there is one suborder, viz. the Corynebacterineae, which houses, next to a large amount of non-pathogenic bacteria, a substantial number of pathogens. It appears that these pathogens reside within the phylogenetic group known as the nocardioform actinomycetes, which encompasses the families Mycobacteriaceae, Nocardiaceae and Corynebacteriaceae (see i.a. chapter 11, titled: Rhodococcus equi: Pathogenesis and Replication in Macrophages, in “Opportunistic Intracellular Bacteria and Immunity”, by Lois J. Paradise et al (eds.), New York, 1999).

During recent years the recognition that the families Mycobacteriaceae, Nocardiaceae and Corynebacteriaceae of the phylogenetic group of nocardioform actinomycetes are very closely related families within the suborder of the Corynebacterineae, has been confirmed (see also University of California, San Diego, Outline of Senior Project, Marelle L. Yehuda, Jun. 2, 2005). It has also become clear that in particular the pathogenic bacteria in this group, at least the ones for which no adequate prophylactic treatment is available (such as for example Mycobacterium tuberculosis, Nocardia seriolae and Rhodococcus equi), have an important property in common: infection typically occurs via skin or mucous membrane, followed by dissemination of the bacteria within macrophages and replication within these macrophages (see i.a. Microbes and Infection 7, 2005, 1352-1363; Proceedings of the National Academy of Sciences, Jun. 7, 2005, Vol. 102, no 23, pp 8327-8332; Nature Medicine 13, 282-284, 2007; Transplantation Proceedings, Volume 36, Issue 5, June 2004, pp 1415-1418). Indeed macrophages are at the frontline of host immune defense against microbial infections, but unlike bacteria that depend on the avoidance of phagocytosis to survive in the host, the currently contemplated pathogenic bacteria within this group target macrophages to survive and even replicate in the host. The present invention is concerned with these bacteria that have the ability to survive within macrophages of a human or animal, and in connection with the current invention will be referred to as macrophage surviving nocardioform actinomycetes.

Apparently, the macrophage surviving nocardioform actinomycetes have evolved to evade critical functions of a human defense against microbes. In particular Mycobacterium tuberculosis, the causative microbe of tuberculosis, is a species that has successfully exploited macrophages as its primary niche in vivo, but other bacterial species that belong to the group of nocardioform actinomycetes, including Mycobacteriaceae, Nocardiaceae and Corynebacteriaceae, have adopted the same strategy. These are for example Mycobacterium ulcerans that causes Buruli ulcer, Mycobacterium avium paratuberculosis that causes Johne's disease in cattle and which is linked to Crohn's disease in humans, Mycobacterium bovis that causes bovine tuberculosis, Mycobacterium avium which is related to opportunistic infection of immunocompromised subjects such as AIDS-patients, Nocardia seriolae and Nocardia farcinia that cause nocardiosis in fish, Nocardia asteroides which causes infection in renal transplant recipients, Rhodococcus equi (formerly known as Corynebacterium) that causes pneumonia in foals and which is also connected to opportunistic infections in immunocompromised subjects, Corynebacterium pseudotuberculosis that causes abscesses, i.a. in the lungs, in sheep, goats, horses and occasionally also in humans, etc. All of these bacterial species have in common the ability to survive within macrophages, infect them and replicate within this type of host cell.

This typical property seriously hampers the treatment for disorders (in this specification the term “disorder” is used as an equivalent for “disease”) arising from an infection with a bacterium that belongs to the group of macrophage surviving nocardioform actinomycetes. In particular, tuberculosis caused by infection with Mycobacterium tuberculosis is a leading cause of mortality from bacterial infection, latently infecting a third of the world's population and killing 2-3 million people each year. After years in decline, Mycobacterium tuberculosis infections are increasing, largely due to two lethal developments: the association of tuberculosis with HIV-infected individuals and the emergence of multidrug-resistant (MDR) strains of Mycobacterium tuberculosis.

The current standard chemotherapy for tuberculosis involves a 6-month treatment program and a cocktail of drugs: an initial 2-month treatment with 4 drugs (isoniazid (INH), rifampin (RIF), pyrazinamide, and ethambutol) followed by an additional 4-month treatment with INH and RIF. The inadequacies of this chemotherapy include its toxicity, poor patient compliance with the lengthy treatment, and ineffectiveness against MDR strains. Chemotherapy against MDR—Mycobacterium tuberculosis involves more toxic drugs, may last up to two years and is expensive with the additional complication of even poorer patient compliance.

Accordingly, a long felt need exists for safe and effective methods for treatment of tuberculosis and other diseases caused by a bacterium that belongs to the group of nocardioform actinomycetes and providing pharmaceutical compositions for the treatment of a disease caused by a bacterium that belongs to the group of nocardioform actinomycetes.

### SUMMARY OF THE INVENTION

The present invention provides in a first embodiment a pharmaceutical composition for use in the treatment of a disease caused by a bacterium that belongs to the group of nocardioform actinomycetes, said composition comprising an effective amount of a compound selected from compound I, (+)-compound II, (−)-compound II, compound III, or mixtures thereof:

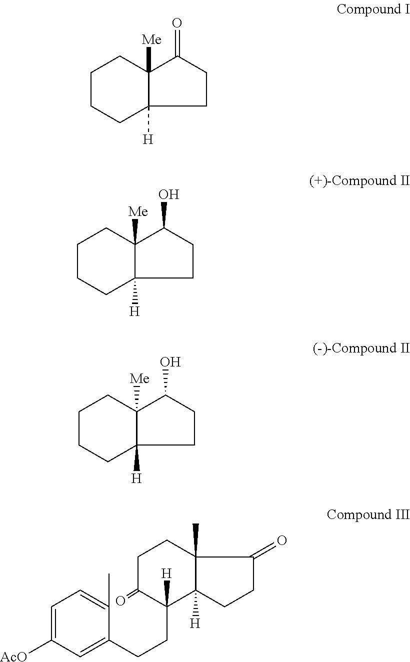

In a second embodiment the present invention provides a method for treating a subject suffering from a disease caused by a bacterium which belongs to the group of nocardioform actinomycetes, said method comprising administering to the subject an effective amount of a compound selected from compound I, (+)-compound II, (−)-compound II, compound III, or mixtures thereof.

Finally, the present invention provides a novel compound (+)-(1S,3aR,7aS)-7a-methyl-1H-octahydroinden-1-ol

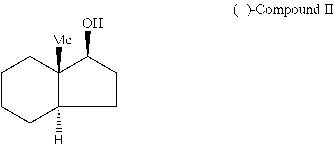

### BRIEF DESCRIPTION OF THE DRAWINGS

FIG. 1 illustrates growth of gene inactivated mutants of R. erythropolis RG8-37 on glucose mineral agar medium supplemented with 0.01% (w/v) HIL. No growth indicates the formation of the growth inhibitor, whereas growth indicates that the introduced gene inactivation blocked inhibitor synthesis. Gene inactivation of fadD3 (strain RG47), ipdF (strain RG48) and fadE30 (strain RG8-37/pAR1818) in strain RG8-37, but not fadE31 (strain RG45) and echA13 (strain RG46), releases growth inhibition caused by the presence of HIL. This indicates that HIL is further metabolized into a toxic compound which involves at least fadD3, ipdF and fadE30 in strain RG8-37.

FIG. 2 illustrates chemical structures of test compounds used.

FIG. 3A illustrates growth curves of wild type strain Rhodococcus erythropolis SQ1 on glucose mineral medium in the absence (diamonds) and presence of 0.01% of (−)-compound II (stars), 0.01% of compound III (circles), 0.01% of racemic compound II (squares), or 0.01% of (+)-compound II (triangles).

FIG. 3B illustrates growth curves of wild type strain Mycobacterium smegmatis mc²155 on glucose mineral medium in the absence (diamonds) and presence of 0.01% of racemic compound II (squares) or compound III (circles).

### DETAILED DESCRIPTION OF THE INVENTION

It has been described in literature that cholesterol metabolism plays a crucial role in the survival of nocardioform actinomycetes in macrophages and is an important virulence factor (Proceedings of the National Academy of Science, Feb. 6, 2007, vol. 104, no. 6, pp 1947-1952).

Various genes of Mycobacterium tuberculosis involved in cholesterol catabolism are specifically expressed during growth in macrophages (SCHNAPPINGER et al 2003. J. Exp Med 198:693-704) and a subset of these genes are essential for survival in macrophages (RENGARAJAN et al 2005. Proc. Natl. Acad. Sci. USA 102:8327-32). Several genes have been implicated in preventing acidification of the Mycobacterium tuberculosis-containing phagosome (PETHE et al 2004. Proc. Natl. Acad. Sci. USA 101:13642-13647).

Macrophage plasma membrane cholesterol has a role in internalization of mycobacteria by macrophages, and sequestration of cholesterol in an in vitro macrophage model inhibits uptake and phagocytosis of mycobacterial. (GATFIELD et al 2000. Science 288: 1647-1 650; PEYRON et al 2001. J. Immunol. 165:5186-5191).

Catabolic studies in a mycobacterial strain indicated that cholesterol is degraded via 4-androstene-3,17-dione (4-AD) with side chain degradation at C-17 likely preceding steroid ring degradation (SMITH et al 1993. Appl. Environ. Microbiol. 59:1425-1429).

It has also been suggested that cholesterol catabolism provides logical targets for novel therapeutic agents to combat disease causing strains, i.e. drugs for treatment after infection has occurred. Indeed, when applying hindsight there is other supporting evidence for the established fact that for all macrophage surviving nocardioform actinomycetes, cholesterol catabolism plays a role in the survival and persistence of the bacteria in host macrophages. For example, from chapter 11 (titled: Rhodococcus equi: Pathogenesis and Replication in Macrophages) in “Opportunistic Intracellular Bacteria and Immunity”, by Lois J. Paradise et al (eds.), New York, 1999) it is known that there are great similarities in the clinical symptomatology between infections caused by several nocardioform actinomycetes and cholesterol oxidase was determined to be an enzymatic component of virulence factors. In Veterinary Microbiology, Volume 56, Issue 3-4, June 1997, 269-276 it is shown that Corynebacterium pseudotuberculosis is involved in the cholesterol oxidase process together with Rhodococcus equi.

WO 2007/118329 discloses enzymes involved in cholesterol degradation in Mycobacterium tuberculosis. Some of these enzymes are essential for growth of Mycobacterium tuberculosis in the macrophage and participate in oxygenolytic cleavage of the rings of cholesterol. Described are substrate analogues and inhibitors of such enzymes which may be used for the treatment of mycobacterial infections including tuberculosis.

As is commonly known, methylhexahydroindanedione propionate (HIP; 3aα-H-4α(3′-propionic acid)-7aβ-methylhexahydro-1,5-indanedione) and 5-hydroxy-methylhexahydroindanone propionate (HIL; 3aα-H-4α(3′-propionic acid)-5α-hydroxy-7aβ-methylhexahydro-1-indanone-δ-lactone) are formed during the degradation of cholesterol by actinobacteria, including the macrophage surviving nocardioform actinomycetes. Recently, an operon (called ipdAB: indanedione proprionate degradiation Alfa+Beta) has been identified in bacterial species that belong to the suborder of Corynebacterineae. This ipdAB operon encodes the α and β subunit of a transferase that is involved in HIP and HIL degradation (see co-pending International Patent application PCT/EP2008/060844, filed 19 Aug. 2008, based on a US priority application filed 21 Aug. 2007). Inactivation of the ipdAB genes in Rhodococcus, encoding ipdAB, has shown to have marked inhibitory effects and effectively blocks cholesterol metabolism and 9α-hydroxylation of 4-androstene-3,17-dione (AD).

Based on these results it was stipulated that HIP or HIL or metabolites derived thereof might inhibit growth of Rhodococcus indicating the formation from these two compounds of a natural growth inhibitor acting as an antibiotic.

Surprisingly, it was found that the compounds I, (+)-II, (−)-II, and III

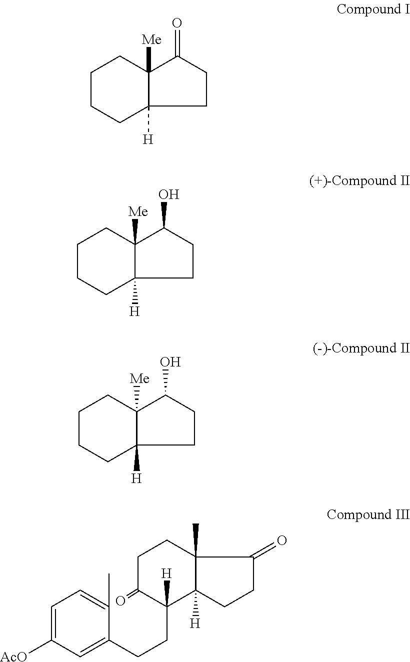

show inhibitory activity in wild type Rhodococcus erythropolis SQ1.

In particular (+)-compound II resulted in the most effective growth inhibition.

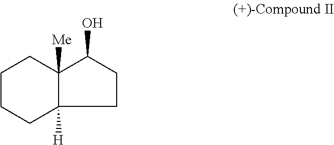

IpdAB homologous genes are present in other nocardioform actinomycetes, including M. tuberculosis. As mentioned above, it was reported by others in the literature that knocking out certain genes in M. tuberculosis resulted in its inability to survive in macrophages and it was concluded that these might be pathogenicity genes (RENGARAJAN et al 2005. Proc. Natl. Acad. Sci. USA 102:8327-32). The function of these genes in M. tuberculosis was not clear to RENGARAJAN et al., but the present inventors were able to recognize that they have a sequence similar to that of ipdAB (called rv3551 and rv3552 genes, respectively).

Accordingly, the present invention provides in a first embodiment a pharmaceutical composition for use in the treatment of a disease caused by a bacterium that belongs to the group of nocardioform actinomycetes, said composition comprising an effective amount of a compound selected from compound I, (+)-compound II, (−)-compound II, compound III, or mixtures thereof:

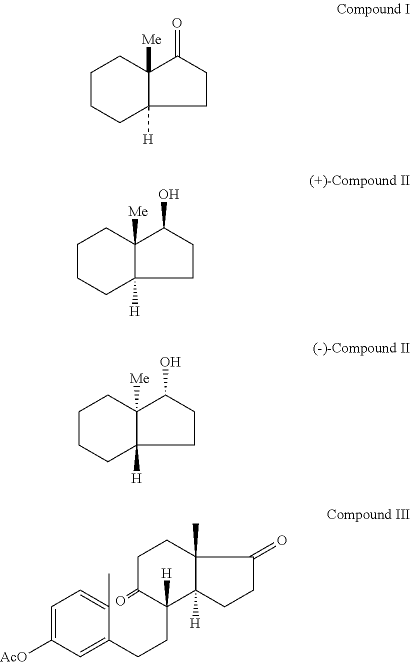

In a second embodiment the present invention provides a method for treating a subject suffering from a disease caused by a bacterium which belongs to the group of nocardioform actinomycetes, said method comprising administering to the subject an effective amount of a compound selected from compound I, (+)-compound II, (−)-compound II, compound III, or mixtures thereof.

Alternatively, the disease is caused by a bacterium of one of the family Mycobacteriaceae, Nocardiaceae or Corynebacteriaceae. More preferably, the disease is caused by a bacterium of one of the genera Mycobacterium, Nocardia, Rhodococcus, and Corynebacterium. Most preferably, the disease is caused by a bacterium of one of the species Mycobacterium tuberculosis, Mycobacterium ulcerans, Mycobacterium bovis, Mycobacterium avium, Mycobacterium avium paratuberculosis, Nocardia seriolae, Nocardia farcinia, Nocardia asteroides, Rhodococcus equi, or Corynebacterium pseudotuberculosis.

In an alternative embodiment, diphtheria, tuberculosis in cattle, equine tuberculosis, or tuberculosis in humans are diseases which can be treated by the pharmaceutical compositions of the present invention.

Preferably, the pharmaceutical composition comprises (+)-compound II

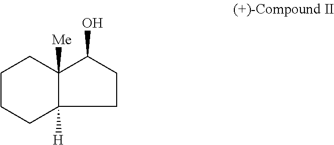

Racemic compound I and its preparation is known i.a. from Snider B. et al, J. Am. Chem. Soc., 1983, 105, 2364-2368.

Racemic compound II has been published by Müller, M. et al, Tetrahedron, 1981, 37, 257. Racemic compound II can be prepared by reduction of racemic compound I under the influence of sodiumborohydride in ethanol.

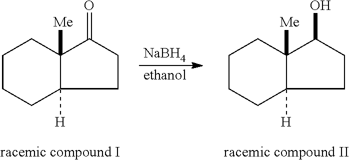

For racemic compound II, the following nomenclature is suggested: rac-(1β,3aα,7aβ)-7a-methyl-1H-octahydroinden-1-ol.

The (+)- and (−)-enantiomers of compound II are novel compounds. They can be obtained by separation of the racemic mixture via preparative chiral HPLC of the corresponding o-nitrobenzoate ester (rac-4).

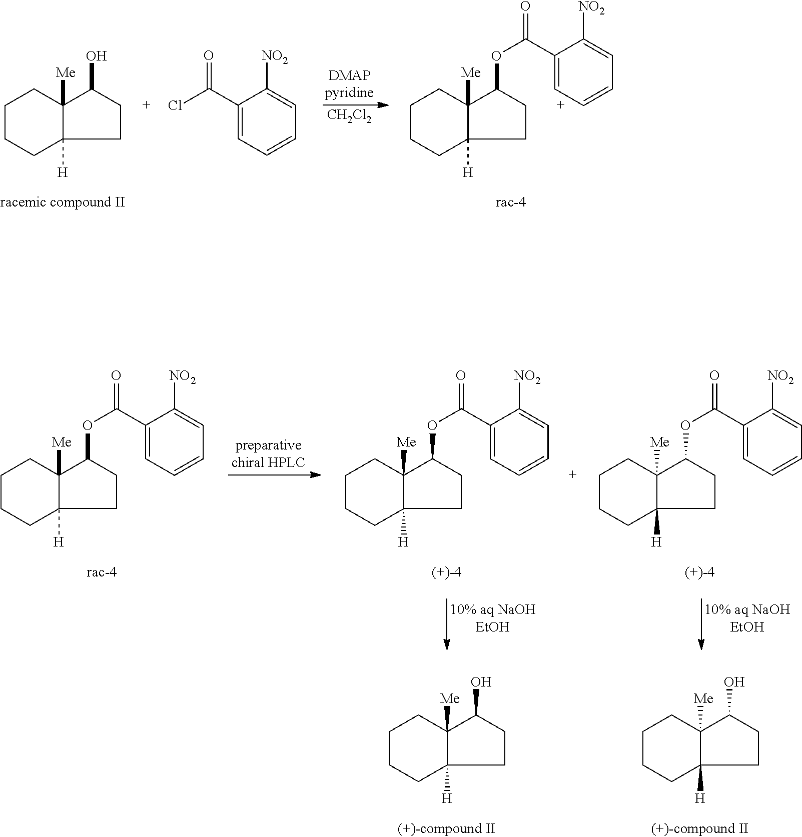

After isolation of the individual enantiomers, the ester groups are saponified under the influence of aqueous sodium hydroxide in ethanol. In this manner, the enantiomers of compound II can be obtained in pure form (enantiomeric excess >95%).

The absolute configuration of both enantiomers of compound II was determined by ¹H-NMR and ¹³C-NMR studies, following the methoxyphenylacetic (MPA) ester-approach as described by Latypoc, Sh. K. et al, J. Org. Chem., 1996, 61, 8569. The (−)-enantiomer of compound II was transformed into the corresponding methoxyphenylacetic esters 5, using both (S)-MPA and (R)-MPA. The absolute configuration of the (−)-enantiomer was determined to be (1R,3aS,7aR).

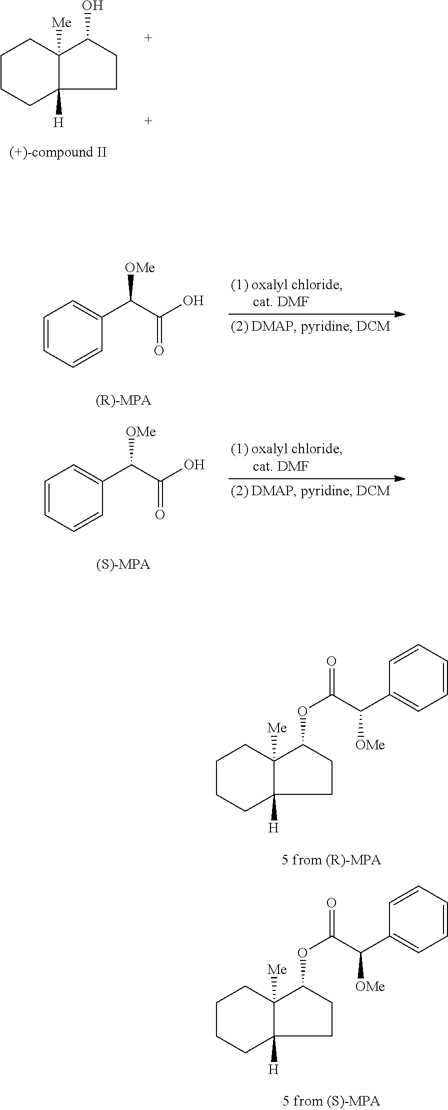

(−)-(1R,3aS,7aR) 7a-methyl-1H-octahydroinden-1-ol is the less active enantiomer.

(+)-(1S,3aR,7aS)-7a-methyl-1H-octahydroinden-1-ol corresponds to the active enantiomer.

Compound III and its preparation is known i.a. from Takeda K. et al., Chem. Pharm. Bull., 23 (11), 1975, pp. 2711-2727.

As used herein, a “subject” refers to a human or other animal.

Compounds of the invention can be provided alone or in combination with other compounds (for example, nucleic acid molecules, small molecules, peptides, or peptide analogues), in the presence of a liposome, an adjuvant, or any pharmaceutically acceptable carrier, in a form suitable for administration to mammals, for example, humans, cattle, sheep, horses, etc. If desired, treatment with a compound according to the invention may be combined with more traditional and existing therapies for a disease caused by a bacterium that belongs to the group of nocardioform actinomycetes, e.g., tuberculosis. For example, treatment with one or more of compound I, (+)-compound II, (−)-compound II, or compound III may be combined with one or more of isoniazid (INH), rifampin (RIF), pyrazinamide or ethambutol.

Compositions comprising one or more of compound I, (+)-compound II, (−)-compound II, or compound III according to any of the various embodiments of the invention, may be administered as a dose from about 0.1 ug/kg to about 20 mg/kg (based on the mass of the subject), or any amount there between, for example from about 1 ug to about 2000 ug/ml or any amount there between, about 10 ug to about 1000 ug or any amount there between, or about 30 ug to about 1000 ug or any amount there between. For example, a dose of about 0.1, 0.5, 1.0, 2.0, 5.0, 10.0 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, 5000, 10000, 20000 ug, or any amount there between may be used. In alternative embodiments, a suitable dosage range may be any integer from 0.1 nM-0.1 M, 0.1 nM-0.05M, 0.05 nM-1 5 μM or 0.01 nM-10 μM.

An “effective amount” of a compound as used herein refers to the amount of compound required to have a prophylactic, palliative or therapeutic effect when administered to a subject. For therapeutic or prophylactic compositions, the compounds may be administered to an individual in an amount sufficient to stop or slow a disease caused by a bacterium that belongs to the group of nocardioform actinomycetes, e.g., tuberculosis. A “therapeutically effective amount” refers to an amount effective, at dosages and for periods of time necessary, to achieve the desired therapeutic result, such as elimination or reduction in the severity of a disease caused by a bacterium that belongs to the group of nocardioform actinomycetes, e.g. tuberculosis. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as prevention of a disease caused by a bacterium that belongs to the group of nocardioform actinomycetes, e.g. tuberculosis. Typically, a prophylactic dose is used in subjects prior to or at an earlier stage of disease, so that a prophylactically effective amount may be less than a therapeutically effective amount.

Mixed with pharmaceutically suitable carriers, e.g. as described in the standard reference, Gennaro et al., Remington's Pharmaceutical Sciences, (18th ed., Mack Publishing Company, 1990, see especially Part 8: Pharmaceutical Preparations and Their Manufacture) the compounds may be compressed into solid dosage units, such as pills, tablets, or be processed into capsules or suppositories. By means of pharmaceutically suitable liquids the compounds can also be applied in the form of a solution, suspension, emulsion, e.g. for use as an injection preparation, or as a spray. For making dosage units, e.g. tablets, the use of conventional additives such as fillers, colorants, polymeric binders and the like is contemplated. In general any pharmaceutically acceptable carrier which does not interfere with the function of the active compounds can be used. Suitable carriers with which the compositions can be administered include lactose, starch, cellulose derivatives and the like, or mixtures thereof, used in suitable amounts. Thus, compositions of the invention can be formulated for any route of administration.

The invention will be further explained using the following examples describing specific embodiments of the present invention.

### Experimental Procedures

### Culture Media and Growth Conditions

Rhodococcus erythropolis SQ1 wild type and mutant strains were grown at 30° C. (200 rpm) in LBP medium consisting of 1% Bacto-Peptone (BD), 0.5% Yeast Extract (BD) and 1% NaCl (Merck). Mycobacterium smegmatis mc²155 wild type and mutant strains (Snapper et al., 1990, Mol. Microbiol. 4:1911-1919) was grown at 37° C. (200 rpm) in BBL trypticase soy broth (TSB; BD) supplemented with 0.05% Tween80. Mineral medium (MM, pH 7.2) contained K₂HPO₄ (4.65 g/l), NaH₂PO₄.H₂O (1.5 g/l), Na-acetate (2 g/l), NH₄Cl (3 g/l), MgSO₄.7H₂O (1 g/l), and Vishniac stock solution (1 ml/l). MM medium was supplemented with different carbon and energy sources: glucose (20 mM), glycerol (20 mM), AD (0.5 g/l), or HIL (0.5 g/l). Vishniac stock solution was prepared as follows (modified from Vishniac and Santer (1957) Bacteriol Rev 21: 195-213): EDTA (10 g/l) and ZnSO₄.7H₂O (4.4 g/l) were dissolved in distilled water (pH 8 using 2 M KOH). Then, CaCl₂.2H₂O (1.47 g/l), MnCl₂.7H₂O (1 g/l), FeSO₄.7H₂O (1 g/l), (NH₄)₆Mo₇O₂₄.4H₂O (0.22 g/l), CuSO₄.5H₂O (0.315 g/l) and CoCl₂.6H₂O (0.32 g/l) were added in that order at pH 6 and finally stored at pH 4. For growth on solid media Bacto-agar (15 g/l; BD) was added. HIL stock solutions (100 mg/ml) were prepared in 1 M NaOH.

### AD Bioconversion

Pre-cultures of R. erythropolis RG8-37 parent and mutant strains were grown in 25 ml LBP medium for 24-36 hours at 30° C. and used to inoculate 50 ml liquid glucose mineral medium (1:100). The cultures were grown for 40 hours at 30° C. at which point AD (0.5 g/l) and HIL (100 mg/l) were added. The bioconversion of AD into 9OHAD was followed by sampling every 2 hours. Steroid content of the samples was analyzed by high-performance liquid chromatography (HPLC). Culture samples (0.5 ml) were mixed with 2 ml of 80% methanol solution and filtered (0.2 μm) prior to analysis by HPLC-UV₂₅₄ₙₘ. HPLC was performed on a C18 column (250×4.6 mm; Alltech, Deerfield, USA, 35° C.) using a mobile phase consisting of methanol:water (80:20) at a flow rate of 1 ml/min. Percentage of AD conversion was calculated as (9OHAD peak area/AD peak area)/(9OHAD peak area+AD peak area)*100%.

Growth Inhibition Screening-Assays R. erythropolis and M. smegmatis

Inhibition of growth of R. erythropolis strains was tested on glucose mineral agar plates. Growth inhibition of R. erythropolis strains and M. smegmatis strains was also tested in glucose or glycerol mineral liquid media containing the test compound at a final concentration of 100 mg/l. Pre-cultures (25 ml) of R. erythropolis st rains and M. smegmatis strains were grown in LBP or TSB+0.05% Tween80, respectively, for 24-36 hours and used to inoculate (1:100) MM medium (50 ml) supplemented with glucose (20 mM) or glycerol (20 mM). Test compounds were added to the medium prior to inoculation in a final concentration of 100 mg/L from stock solutions (100 g/L) dissolved in 1 M NaOH (HIL) or methanol (all other compounds tested). Cell growth was followed by measuring cell culture turbidity at 600 nm for several days.

Gene Inactivation of fadD3, echA13, fadE31 and ipdF in R. erythropolis RG8-37

Unmarked gene deletion mutant strains of strain RG8-37 were essentially constructed using the sacB counter selection method as reported previously (van der Geize et al. (2001) FEMS Microbiol Lett 205:197-202). Gene disruption was essentially performed as described (Van der Geize et al. (2000) Appl Environ Microbiol 66: 2029-2036). In order to delete fadE31, plasmid pAR1812 was constructed as follows. Plasmid pAR1800 was digested with ScaI/BgIII, treated with Klenow and self-ligated, resulting in pAR1811. Subsequently, pAR1811 was cut with SphI/HindIII and treated with Klenow, and a 3.2 kb DNA fragment carrying a 0.4 kb deletion in fadE31 was ligated into SphI/HindIII digested pK18mobsacB, yielding pAR1812. R. erythropolis mutant strain RG45 was subsequently made using pAR1812 carrying a fadE31 gene deletion. The fadE31 deletion was confirmed by PCR using forward primer 5′ ACGCCACAACCGCATTCCGTGA and reverse primer 5′ TCGTTGGTGCCTGCGTAGATCG resulting in a 685 bp product for the fadE31 mutant as compared to 1,085 bp for the wild type gene. For echA13 gene deletion, plasmid pAR1816 was constructed as follows. A 2.9 kb Acc65I DNA fragment was treated with T4 DNA polymerase and blunt ligated into SmaI digested pK18mobsacB. The resulting plasmid, pAR1815, was subsequently digested with BstXI, treated with T4 DNA polymerase and self ligated, yielding pAR1816 carrying a 0.45 kb deletion of echA13. R. erythropolis mutant strain RG46 was subsequently made using pAR1816 carrying an echA13 gene deletion. The echA13 deletion was confirmed by PCR using forward primer 5′ GCAGGCAACGGACCTCACTTCA and reverse primer 5′ CTAGTTTGTTCCTTCCTGCGGT resulting in a 239 bp product for the echA13 mutant as compared to 699 bp for the wild type gene. For fadD3 deletion, plasmid pAR1817 was constructed as follows. A 3.7 kb SpeI DNA fragment of pAR1800 carrying fadD3 was ligated into pBluescript (II) KS, yielding pAR1813. A 0.8 kb internal DNA fragment of fadD3 was removed by SgrAI restriction of pAR1813 followed by self-ligation. The resulting plasmid pAR1814 was digested with SpeI and a 3 kb DNA fragment was ligated into XbaI digested pK18mobsacB, yielding pAR1817. R. erythropolis mutant strain RG47 was subsequently made using pAR1817 carrying a fadD3 gene deletion. The fadD3 deletion was confirmed by PCR using forward primer 5′ CCGACTGACCTTCGCACAGCTA and reverse primer 5′ ATGCCGATGGCAGCAGACTCGT resulting in a 489 bp product for the fadD3 mutant as compared to 1,248 bp for the wild type gene.

For fadE30 gene disruption, pAR1818 was constructed by ligating a Klenow treated 0.64 kb BamHI/XmnI blunt-end DNA fragment of pAR1800, harbouring an internal gene fragment of fadE30, into SmaI digested pK18mobsacB. The R. erythropolis fadE30 disruption mutant strain RG8-37/pAR1818 was subsequently made by introduction of pAR1818 into strain RG8-37.

Construction of Mycobacterium smegmatis ΔipdAB

For construction of the ΔipdAB mutant of M. smegmatis strain mc²155, the non-replicative plasmid pK18-ipdABsmeg was mobilized to M. smegmatis by electrotransformation essentially as described (Jacobs et al. (1991) Methods Enzymol 204: 537-555). Briefly, cell cultures (250 ml) were grown at 37° C. in TSB medium+0.05% Tween80 until OD₆₀₀ reached 0.8, put on ice for one and a half hour and centrifuged (10 min at 5,000×g) to pellet the cells. Cell pellets were washed twice with distilled water and resuspended in a final volume of 1 ml 10% glycerol and divided into 200 μl aliquots. MilliQ-eluted plasmid DNA (5-10 μl; GenElute Plasmid Miniprep Kit, Sigma-Aldrich) was added to 200 μl cells in 2 mm gapped cuvettes. Electroporation was performed with a single pulse of 12.5 kV/cm, 10000 and 25 μF. Electroporated cells were gently mixed with 1 ml TSB+0.05% Tween80 and allowed to recover for 5 h at 37° C. and 200 rpm. Aliquots (200 μl) of the recovered cells were plated onto selective TSB+0.05% Tween80 agar containing kanamycin (10 μl/ml). Several transformants were obtained after 4-5 days of incubation at 37° C. One kanamycin resistant transformant was grown for 2 days at 37° C. non-selectively in TSB medium containing 0.05% Tween80 and subsequently plated onto TSB agar plates containing 2% sucrose to select for kanamycin sensitive (Km^{S}) and sucrose resistant (Suc^{R}) double-recombinants by sacB counter-selection. Colonies appearing after 3 days of incubation were replica streaked onto TSB agar and TSB agar supplemented with kanamycin (10 μl/ml) to select for Km^{S}/Suc^{R} colonies. Genuine Km^{S}/Suc^{R} colonies were further checked by colony PCR for the presence of the ipdAB gene deletion with forward primer ipdABMsmegcont-F ACGCCAGCTACCGCATGGAA and reverse primer ipdABMsmegcont-R ATCACCTCGCGCAGCAGCTT. Genomic DNA was isolated from three potential ipdAB mutants and PCR analysis using the aforementioned primers confirmed the presence of the ipdAB gene deletion (273 bp) and the absence of the wild type ipdAB genes (1697 bp) in all three mutants. One ipdAB mutant strain was chosen for further work and designated M. smegmatis ΔipdAB.

### Synthesis of the (+)- and (−)-Enantiomers of Compound II

### Synthesis of Racemic 4

Racemic compound II (200 mg, 1.30 mmol) was reacted with o-nitrobenzoyl chloride (481 mg, 2.59 mmol), pyridine (210 μl, 2.59 mmol) and DMAP (15.8 mg, 0.13 mmol) in dichloromethane. After stirring for 17 h at ambient temperature, the reaction was quenched with 1 M HCl and the product was extracted with ethyl acetate. The combined organic layers were washed with 1 M HCl, aqueous saturated NaHCO₃ solution and brine. The organic layer was dried with Na₂SO₄ and concentrated in vacuo. The crude material was purified by flash chromatography, yielding 300 mg of racemic ester 4 (74% yield).

### Separation of Enantiomers

The ester synthesis was repeated on a scale of 2.2 g. The thus obtained racemic ester was separated using a Chiralcel AD-H column (conditions 1% 2-propanol in heptane, flow 18 mL/min, 30 min, collected at λ=210 nm, collection threshold 10 mV). The two enantiomers were obtained in 0.65 g and 0.55 g respectively (see Table).

| (−)-4 | (+)-4 |
|---|---|
| 0.65 g | 0.55 g |
| Purity (GC) > 95%, | Purity (GC) > 95%, |
| ee > 95% (chiral HPLC) | ee > 95% (chiral HPLC) |
| [α]_{D}²⁰ = −36.2 (c = 1, CHCl₃) | [α]_{D}²⁰ = +41.3 (c = 1, CHCl₃) |

### Removal of the Benzoate Esters

Both enantiomers were subjected to the same saponification methods. A suspension of appr. 600 mg of the ester in 12 mL ethanol was treated with 10% aqueous NaOH (3 mL). The reaction was continued at room temperature and the conversion was monitored by LC/MS. After full conversion, the product was extracted with ethyl acetate. After washing, drying and concentration, the product was obtained (appr. 200 mg).

| (−)-compound II | (+)-compound II |
|---|---|
| 244 mg | 187 mg |
| Purity (GC/MS) > 95% | Purity (GC/MS) > 95% |
| [α]_{D}²⁰ = −18.5 (c = 1, CHCl₃) | [α]_{D}²⁰ = +14.0 (c = 1, CHCl₃) |

### Formation of the MPA Esters

To determine the absolute configuration via NMR studies, the (−)-enantiomer of compound II was transformed into the corresponding methoxyphenylacetic (MPA) ester. For the NMR studies both diastereomers of ester 5 were formed from (S)- and (R)-MPA respectively. To this end, MPA (166 mg, 1.0 mmol) was reacted with oxalyl chloride (0.26 mL, 3.0 mmol) in THF (2 mL) in the presence of a catalytic amount of DMF. After stirring for 2 h at ambient temperature, the reaction mixture was concentrated in vacuo. The thus obtained acid chloride of MPA was dissolved in pyridine and a solution of (−)-compound II (30 mg, 0.19 mmol) in pyridine (1 mL) and a catalytic amount of DMAP were added. After stirring for 17 h at ambient temperature, the reaction mixture was quenched with a citric acid solution. The product was extracted with ethyl acetate. The residue obtained after concentration of the combined organic layers was purified by column chromatography. The product was obtained as a white foam (30 mg, 0.099 mmol, 52% yield). The absolute configuration of the (−)-enantiomer was determined to be (1R,3aS,7aR) via NMR studies. Thus, the absolute configuration of the (+)-enantiomer is (1S,3aR,7aS).

### EXAMPLES

### Metabolism of AD is Necessary for Inhibitor Formation: Construction of ipdAB Gene Deletion Mutant R. erythropolis RG8-37 to Uncouple Steroid Degradation from Inhibitor Formation

To confirm that the inhibitor is formed through AD metabolism in an ipdAB mutant genetic background, an ipdAB gene deletion was made in a R. erythropolis mutant strain (strain RG8) that cannot completely metabolize AD. R. erythropolis strain RG8 is a mutant strain devoid of the 3-ketosteroid Δ1 dehydrogenase (KSTD), encoding genes kstD and kstD2. Strain RG8 is effectively blocked in AD Δ1-dehydrogenation and capable of stoichiometric conversion of AD into 9OHAD due to 3-ketosteroid 9α-hydroxylase activity (WO2001/031050; Van der Geize et al. (2002) Mol Microbiol 45: 1007-1018). 9OHAD cannot be converted further to lower pathway intermediates due to the absence of KSTD activity. Unmarked in-frame gene deletion of ipdA and ipdB in R. erythropolis strain RG8 was achieved using plasmid pAR31 as described previously (co-pending International Patent application PCT/EP2008/060844, filed 19 Aug. 2008). The resulting ipdAB kstD kstD2 mutant strain of R. erythropolis RG8 was designated strain RG8-37. Mutant strain RG8-37 did not grow on MM agar plates supplemented with HIL (MM-HIL) as sole carbon and energy source.

Bioconversion of AD (0.5 g/L) with cell cultures of strain RG8-37 grown in mineral glucose medium revealed that 3-ketosteroid 9α-hydroxylase (KSH) activity was not inhibited: R. erythropolis strain RG8-37 performed AD conversions into 9OHAD with yields of up to 90% within 8 hours. These results indicated that the inhibitor of KSH activity is formed following the degradation of AD in an ipdAB mutant.

Addition of HIL (100 mg/L) to similar AD bioconversions by cell cultures of strain RG8-37 resulted in a marked inhibition of AD 9α-hydroxylation (20% conversion in 8 hours), indicating that the inhibitor is either HIL or a metabolite thereof.

ipdAB Gene Inactivation Results in HIL-Dependent Growth Inhibition on Glucose

In order to examine the effects of HIL on normal cell growth of R. erythropolis strain RG8-37, it was streaked onto glucose mineral agar medium (control) and glucose mineral agar medium supplemented with HIL (0.01% w/v). Strain RG8-37 cells plated onto glucose mineral medium without HIL grew normally, whereas no growth of RG8-37 was observed on glucose agar plates containing HIL after 3 days of incubation. These results indicate that HIL, or a metabolite thereof, has antibiotic properties towards R. erythropolis RG8-37 inhibiting normal cell growth.

Cell cultures of strain RG8-37, inoculated into liquid glucose mineral media containing HIL (100 mg/l), were unable to grow over a period of 96 hours, whereas wild type strain SQ1 grew to stationary phase within 72 hours under the same conditions. This growth experiment was used in subsequent work as an inhibition screening assay to identify compounds and genes involved in formation of the inhibitor.

Identification of Additional Genes of R. erythropolis Involved in HIL Metabolism

HIL and its metabolism play an important role in the formation of the inhibitor. To identify additional genes involved in HIL degradation, UV mutant strain AP18 blocked in growth on MM medium supplemented with HIL was isolated from UV mutagenic treatment of R. erythropolis SQ1 as described previously (co-pending International Patent application PCT/EP2008/060844, filed 19 Aug. 2008). HIL growth deficient mutants (HIL⁻) of R. erythropolis SQ1, growing well on mineral glucose (20 mM) agar plates, were selected following UV mutagenesis. One UV mutant strain, designated AP18, had a glucose⁺/HIL⁻ growth phenotype and was selected for further work. Functional complementation of the mutant HIL⁻ growth phenotype was performed by introducing a genomic library of R. erythropolis (Van der Geize et al. (2002) Mol Microbiol 45:1007-1018) into mutant strain AP18. Colonies that regained the capability to grow on mineral agar medium supplemented with HIL were picked and used for plasmid DNA extraction, resulting in the isolation of plasmid pAR1800. Nucleotide sequence analysis of pAR1800 revealed a total number of 5 intact genes. Thus, one or several of these genes are involved in growth on HIL as sole carbon and energy source. Database similarity searches indicated that these genes were likely functional homologues of fadD3 (rv3561), fadE30 (rv3560c), fadE31 (rv3562), fadE32 (rv3563) and echA13 (rv1935c) found in M. tuberculosis H37Rv (Cole et al. (1998) Nature 393: 537-544).

### Genes Involved in HIL Metabolism are Involved in Inhibitor Formation

To investigate whether the inhibitor is formed through metabolism of HIL, we subsequently constructed several mutants of strain RG8-37. Unmarked gene deletions of fadE31, echA13 and fadD3 were constructed in R. erythropolis RG8-37 using the plasmids pAR1812, pAR1816 and pAR1817, respectively, resulting in mutant strains RG45, RG46 and RG47, respectively. In addition, an ipdF gene deletion strain of strain RG8-37 was constructed as described previously (co-pending International Patent application PCT/EP2008/060844, filed 19 Aug. 2008), designated strain RG48. Finally, a fadE30 gene disruption was made by introducing pAR1818 into strain RG8-37, yielding strain RG8-37/pAR1818.

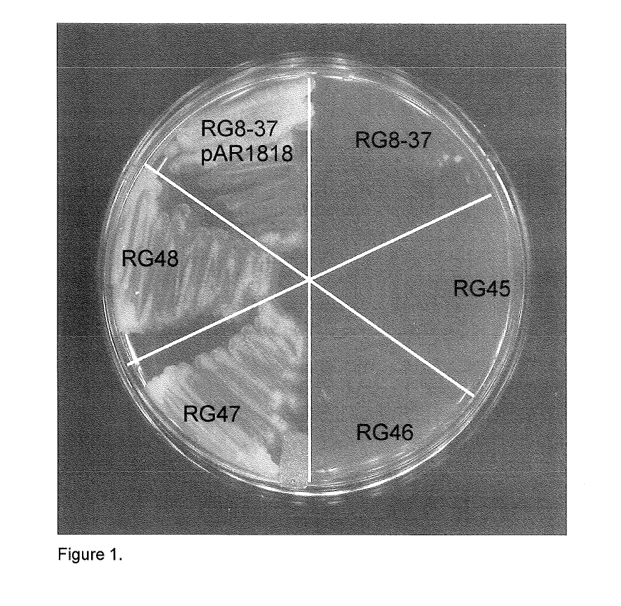

These mutants and parent strain RG8-37 were tested for growth on MM glucose agar medium with and without the addition of HIL 100 (mg/L). All strains grew well on MM glucose medium. Interestingly, growth was also observed with strains RG47, RG48 and RG8-37/pAR1818, whereas growth of strains RG45 and RG46 was still inhibited by the presence of HIL (FIG. 1). These results indicate that fadD3, fadE30, and ipdF, but not fadE31 and echA13, are involved in the HIL dependent formation of the growth inhibitor. The results further clearly show that not HIL, but a metabolite of HIL is responsible for growth inhibition in an ipdAB mutant genetic background.

### Identification of Other Genes Involved in Inhibitor Formation by Random Transposon Mutagenesis

In order to identify additional genes involved in inhibitor formation, transposon mutagenesis of strain RG8-37 was performed using plasmid pKGT452Cβ (Gartemann and Eichenlaub (2001) J. Bacteriol. 183: 3729-3736). The latter was introduced into RG8-37 by electroporation as previously described (Van der Geize et al. (2000) Appl Environ Microbiol 66: 2029-2036). Electroporated cells were plated onto LBP agar medium containing chloramphenicol (40 mg/l) and incubated for 3 days at 30° C. Colonies appearing were replica plated onto glucose mineral agar plates supplemented with HIL (100 mg/l) to select for transposon mutants in which inhibition by HIL was eliminated. Four mutants were obtained that were able to grow on glucose in the presence of HIL.

Chromosomal DNA of these mutants was isolated (Van der Geize et al. (2000) Appl Environ Microbiol 66: 2029-2036) and analyzed by PCR for the presence of cmx (chloramphenicol resistance gene) and bla (ampicillin resistance gene) of pKGT452Cβ. PCR analysis revealed that three out of the four mutants contained both bla and cmx, indicating that genuine transposition had not occurred. Likely, random integration by illegitimate recombination had occurred as reported previously for Rhodococcus (Desomer et al. (1991) Mol. Microbiol. 5:2115-2124). Further analysis revealed that in several transposon mutants chromosomal deletions or rearrangements had occurred which were not further analyzed. One transposon mutant (strain RG8-37B1) was shown to have resulted from the integration of pKGT452Cβ into a single gene. The gene disrupted by pKGT452Cβ was identified as follows. Chromosomal DNA of strain RG8-37B1 was isolated and self-ligated following XhoI digestion. The resulting ligation mixture was used to transform E. coli DH5 α and transformants were selected using chloramphenicol (40 mg/l). An XhoI restriction site does not occur in plasmid pKGT452Cβ. Therefore, all E. coli DH5 α transformants obtained arose from the presence of pKGT452Cβ with additional flanking rhodococcal gene sequences of the gene disruption site. Nucleotide sequence analysis of the plasmid isolated from these E. coli DH5α transformants revealed that a rhodococcal orthologue of rv3559 of M. tuberculosis had been inactivated by pKGT452Cβ insertion. Interestingly, rv3559 in M. tuberculosis is located next and downstream of fadE30 in the M. tuberculosis H37Rv genome. As describe above, fadE30 had already been identified as involved in HIL metabolism and inhibitor formation. Thus, the Rv3559 orthologue in R. erythropolis RG8-37 is yet another gene involved in HIL metabolism and inhibitor formation.

Inactivation of ipdAB in M. smegmatis mc²155 Blocks Growth on HIL

Bioinformatic analysis revealed that all identified genes of R. erythropolis involved in HIL metabolism were conserved in M. tuberculosis H37Rv. To investigate whether inhibition by HIL in an ipdAB genetic background also occurs in mycobacteria, we constructed an ipdAB mutant of Mycobacterium smegmatis mc²155. Unlike M. tuberculosis, M. smegmatis is a fast growing mycobacterial species. Therefore, M. smegmatis is often used as a model organism to study and predict the metabolism of M. tuberculosis.

The ipdA and ipdB genes of M. smegmatis mc ²155 were identified by homology searches and were found to correspond to genes designated MSMEG_{—}6002 and MSMEG_{—}6003, respectively. For the unmarked gene deletion of the ipdAB genes in M. smegmatis mc²155, plasmid pK18-ipdABsmeg was constructed as follows. The upstream (forward primer 5′ TTCGAGATGGCCGCGATCGAAT and reverse primer 5′ ACTAGTGATGGTCATGCCGCTCTCGATA) and downstream (forward primer 5′ ACTAGTCAGGTCGCCGACAACACCTCGT and reverse primer 5′ AAGCTTGAATTCGTCGCCGACGGTGAAG) flanking regions of the ipdAB genes were amplified by PCR using genomic DNA of M. smegmatis mc ²155 as template. The obtained amplicons were ligated into SmaI digested pK18mobsacB (Schafer et al. (1994) Gene 145:69-73), resulting in pK18-ipdABsmegUP and pK18-ipdABsmegDOWN, respectively. A 1.5 kb DNA fragment obtained from BamHI/SpeI digested pK18-ipdABsmegUP was subsequently ligated into pK18-ipdABsmegUP linearized with BamHI/SpeI, resulting in the construction of pK18-ipdABsmeg used for ipdAB gene deletion. An unmarked ipdAB gene deletion mutant of M. smegmatis mc ²155 was constructed using the sacB counter selection system (Pelicic et al. (1996) Mol Microbiol 20: 919-925) as follows.

M. smegmatis ΔipdAB and wild type strain mc²155 were subsequently plated onto mineral agar plates supplemented with HIL (500 mg/l) and incubated at 37° C. Contrary to the wild type strain, the ΔipdAB mutant strain was unable to grow on MM-HIL agar plates, indicating that the ipdAB genes of M. smegmatis are essential for growth on HIL as sole carbon and energy source and indicating that the ipdAB genes have a similar function in mycobacteria and rhodococci.

Cell Growth Inhibition of M. smegmatis ΔipdAB by Addition of HIL

To study the effect of HIL on cell growth of M. smegmatis ΔipdAB, the ipdAB mutant was grown in TSB supplemented with 0.05% Tween80 and grown for 2 days. The pre-culture was used to inoculate (1:500) mineral glucose medium containing 100 and 200 mg/l HIL. Growth inhibition was observed in both cases. Contrary to R. erythropolis RG8-37, addition of HIL did not fully block growth, but delayed the onset of growth for approximately 48 hours.

The inhibition screening was also performed on glucose mineral agar plates containing HIL (200 mg/l). Cell pre-cultures of wild type strain and ΔipdAB mutant strain were plated out and incubated for several days at 37° C. After 3 days of growth, wild type agar plates were confluently grown, whereas no growth appeared with the ipdAB mutant strain. Further incubation of the mutant resulted in the appearance of a small number of spontaneous resistant colonies. Apparently, over time, resistance of ipdAB mutant cells towards HIL had developed, providing an explanation for the delayed growth of this mutant observed in glucose liquid medium supplemented with HIL.

The results indicate that an inhibitor of cell growth is synthesized in the presence of HIL both in Rhodococcus and Mycobacterium species in an ipdAB genetic background.

Identification of HIL Derivatives with Inhibitory Activity Towards Wild Type Rhodococcus

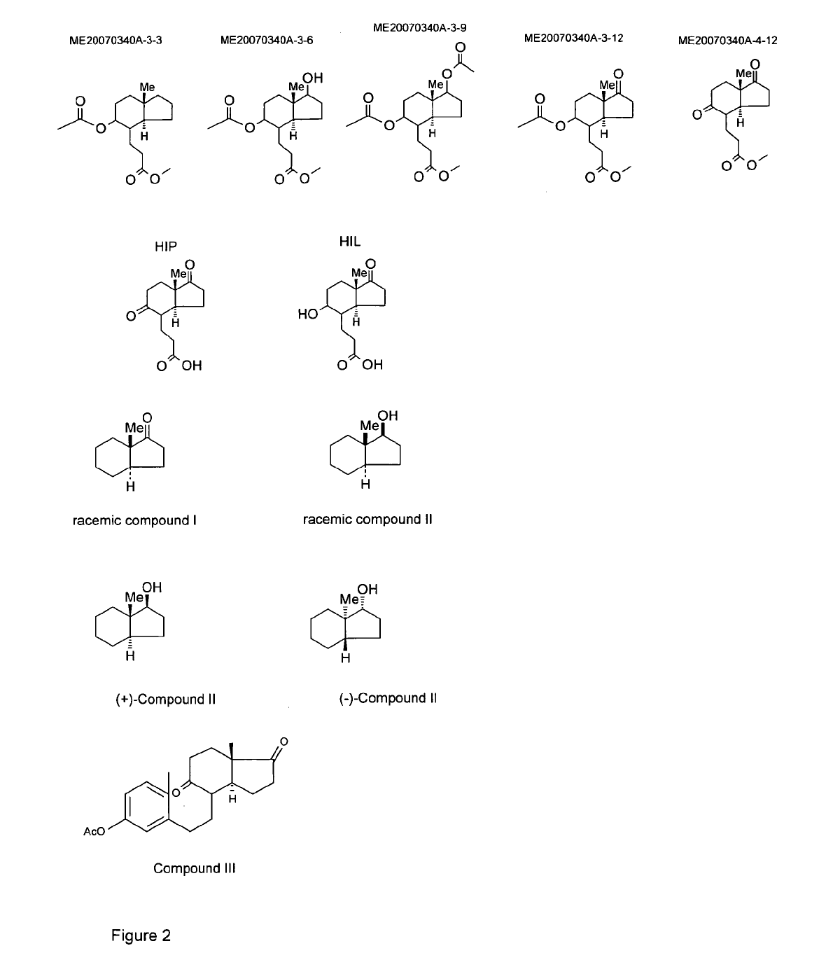

The observed growth inhibition induced in the presence of HIL does not occur with R. erythropolis SQ1 wild type strain. Several derivatives of HIL, harboring the 7a-methyloctahydroinden structure with different substituents, were therefore tested to screen for compounds capable of growth inhibition of wild type R. erythropolis SQ1 on mineral glucose agar plates containing 0.01% (w/v or v/v, depending whether the test compound was solid or liquid) of the test compound (FIG. 2). Growth inhibition of R. erythropolis SQ1 occurred only with compound I, racemic compound II and compound III. Racemic compound II showed the strongest inhibition of growth. Growth inhibition by compound I, racemic compound II and compound III was even more pronounced in mutant strain RG8-37. Thus, the ipdAB genes appear to play a role in the inhibitory effects observed when incubating cells with compound I, racemic compound II and compound III. Bioconversion of AD (0.5 g/L) with cell cultures of strain RG8-37 grown in mineral glucose medium revealed that, contrary to HIL, 3-ketosteroid 9α-hydroxylase activity was not inhibited by racemic compound I and racemic compound II. AD was converted into 9OHAD with yields of up to 90% within 8 hours comparable to controls where no test compound was added. These results suggest that different growth inhibitory metabolites may be formed from compounds containing the 7a-methyloctahydroinden structure.

### Compound II and Compound III Inhibit Growth of Rhodococci and Mycobacteria

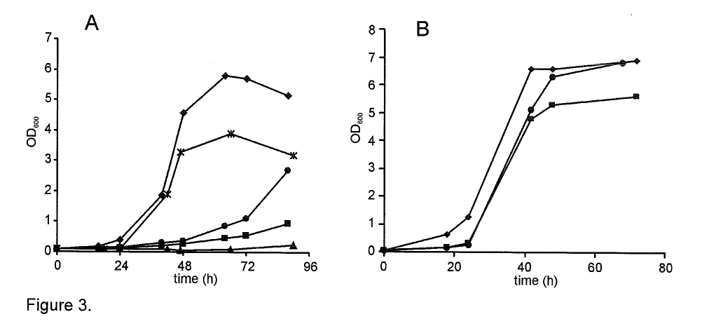

The growth inhibitory activity of racemic compound II and compound III were also tested in glucose mineral liquid cultures of wild type R. erythropolis SQ1 and wild type M. smegmatis mc²155 (FIGS. 3A and B). The addition of 0.01% racemic compound II or 0.01% compound III to such cultures was shown to have a strong inhibitory effect on the growth of R. erythropolis SQ1. The growth of M. smegmatis mc ²155 was also inhibited by these compounds, but to a much lesser extent, indicating differences in metabolism of these test compounds by R. erythropolis SQ1 compared to M. smegmatis mc²155.

The separate enantiomers of compound II were tested in glucose mineral liquid cultures of R. erythropolis SQ1. The addition of 0.01% of (−)-compound II did not inhibit the growth of wild type strain SQ1, although a lower growth yield was obtained (FIG. 3A). The addition of 0.01% of (+)-compound II to cell cultures of wild type strain SQ1 inhibited growth comparable to racemic compound II (FIG. 3). The (+)-(1S,3aR,7aS)-enantiomer is the active constituent of racemic compound II.

1. A pharmaceutical or veterinary composition for use in the treatment of a disease caused by a bacterium that belongs to the group of nocardioform actinomycetes, said composition comprising an effective amount of a compound selected from compound I, (+)-compound II, (−)-compound II, compound III, or mixtures thereof:

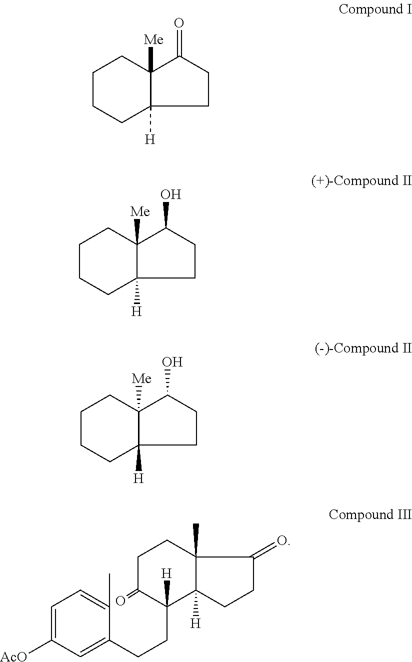

2. The composition of claim 1 comprising an effective amount of (+)-compound II:

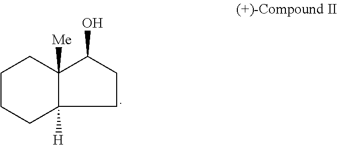

3. The composition of claim 1 comprising in addition one or more pharmaceutical carriers.

4-7. (canceled)

8. A method for treating a subject suffering from a disease caused by a bacterium which belongs to the group of nocardioform actinomycetes, said method comprising administering to the subject an effective amount of a compound
of claim 1 or a composition thereof comprising in addition one or more pharmaceutical carriers.

9. The method according to claim 8 wherein the compound is

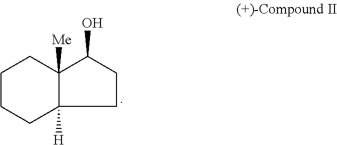

10. The method of claim 8 wherein the disease is caused by a bacterium of one of the family Mycobacteriaceae, Nocardiaceae and Corynebacteriaceae.

11. The method of claim 10 wherein the disease is caused by a bacterium of one of the genera Mycobacterium, Nocardia, Rhodococcus, and Corynebacterium.

12. The method of claim 11 wherein the disease is caused by a bacterium of one of the species Mycobacterium tuberculosis, Mycobacterium ulcerans, Mycobacterium bovis, Mycobacterium avium, Mycobacterium avium paratuberculosis, Nocardia seriolae, Nocardia farcinia, Nocardia asteroides, Rhodococcus equi, or Corynebacterium pseudotuberculosis.

13. The method of claim 8 wherein the disease is diphtheria, tuberculosis in cattle, equine tuberculosis, or tuberculosis in humans.

14. The method of claim 8 wherein the subject is a human.

15. (+)-(1S,3aR,7aS)-7a-methyl-1H-octahydroinden-1-ol [chemical structure]


SEQUENCE LISTING (12 sequences)

SEQ ID NO: 1 - DNA, 22 bp, Artificial
  primer
acgccacaaccgcattccgtga

SEQ ID NO: 2 - DNA, 22 bp, Artificial
  primer
tcgttggtgcctgcgtagatcg

SEQ ID NO: 3 - DNA, 22 bp, Artificial
  primer
gcaggcaacggacctcacttca

SEQ ID NO: 4 - DNA, 22 bp, Artificial
  primer
ctagtttgttccttcctgcggt

SEQ ID NO: 5 - DNA, 22 bp, Artificial
  primer
ccgactgaccttcgcacagcta

SEQ ID NO: 6 - DNA, 22 bp, Artificial
  primer
atgccgatggcagcagactcgt

SEQ ID NO: 7 - DNA, 20 bp, Artificial
  primer
acgccagctaccgcatggaa

SEQ ID NO: 8 - DNA, 20 bp, Artificial
  primer
atcacctcgcgcagcagctt

SEQ ID NO: 9 - DNA, 22 bp, Artificial
  primer
ttcgagatggccgcgatcgaat

SEQ ID NO: 10 - DNA, 28 bp, Artificial
  primer
actagtgatggtcatgccgctctcgata

SEQ ID NO: 11 - DNA, 28 bp, Artificial
  primer
actagtcaggtcgccgacaacacctcgt

SEQ ID NO: 12 - DNA, 28 bp, Artificial
  primer
aagcttgaattcgtcgccgacggtgaag